(19) United States
(12) Patent Application Publication
(10) Pub. No.: US 2010/0154803 A1
(43) Pub. Date: Jun. 24, 2010

(54) COVER FOR BREAST DURING IMPLANT SURGERY, AND METHODS OF PERFORMING BREAST IMPLANT SURGERY

(76) Inventor: Randal Haworth, Los Angeles, CA (US)

Correspondence Address: CERMAK KENEALY VAIDYA & NAKAJIMA LLP, 515 E. BRADDOCK RD, ALEXANDRIA, VA, 22314, US

(21) Appl. No.: 12/631,983

(22) Filed: Dec. 7, 2009

Related U.S. Application Data
(60) Provisional application No. 61/121,656, filed on Dec. 11, 2008.

(51) Int. Cl. A61B 19/08

(52) U.S. Cl. 128/856

(57) ABSTRACT

A cover useful during invasive surgery of the breast includes an adhesively backed sheet and a closed tube that extends from the sheet. The tube is evaginated into an incision in the breast, the closed end of the tube is ruptured, and a breast implant can be inserted through the tube into the breast tissue. Methods can inhibit bacterial infection of the breast tissues.

This application claims priority under 35 U.S.C. §119 to U.S. Provisional application No. 61/121,656, filed 11 Dec. 2008, the entirety of which is incorporated by reference herein.

### BACKGROUND

1. Field of Endeavor

The present invention relates to devices, systems, and processes useful as a cover for a breast, and more specifically to a cover useful during breast implant surgeries.

2. Brief Description of the Related Art

Hundreds of thousands of breast implant surgeries are performed per year in the U.S. While many are not problematic, post-operative complications sometimes arise, in some cases well after the surgery. More specifically, it has been noted that encapsulation of the breast implant occurs, in which the breast tissue surrounding the implant, or capsule, shrinks, causing the breast tissue to tighten around the implant. Excessive shrinkage and tightening of this tissue is generally undesirable, often resulting in pain, distortion of the breast shape, and an unnatural feel to the breast.

It has also been found that a significant cause of encapsulation is a bacterial infection of the breast tissue, which results in scar tissue formation around the implant. It has also been found that in nearly 90% of cases, the skin adjacent the surgically formed access point in the breast, through which the implant is inserted into the breast during implantation surgery, carries bacteria.

During a typical breast implant surgery, a surgeon makes an incision in the breast tissue, often adjacent to the areola, and forms a cavity or pocket in the breast tissue in which the implant is to be inserted. Because of the presence of bacteria on the adjacent skin, and potentially in the breast tissue itself, it has been common practice for surgeons to attempt to perform a ‘touchless’ procedure, that is, a breast implantation procedure involving as little manipulation of the breast tissue as possible, to minimize the introduction of bacteria into the cavity. It has also been proposed for surgeons to place an open-ended, flexible plastic tube into the breast tissue through the surgically formed opening, through which the surgeon is to push the implant. The use of such devices has not been widely adopted, at least in part because the tube is very difficult to manipulate, twists, bunches, and is difficult to manage during implant insertion.

There thus remains an unmet need for systems and methods which can assist surgeons in reducing the occurrence of bacterial infections subsequent to breast implant surgery, and reducing breast implant encapsulation.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a perspective view of an exemplary breast cover embodying principles of the present invention;

FIG. 2 illustrates a top plan view, taken at line A-A in FIG. 1; and

FIGS. 3a-3c illustrate three steps in an exemplary method in accordance with principles of the present invention.

### DETAILED DESCRIPTION OF EXEMPLARY EMBODIMENTS

Referring to the drawing figures, like reference numerals designate identical or corresponding elements throughout the several figures.

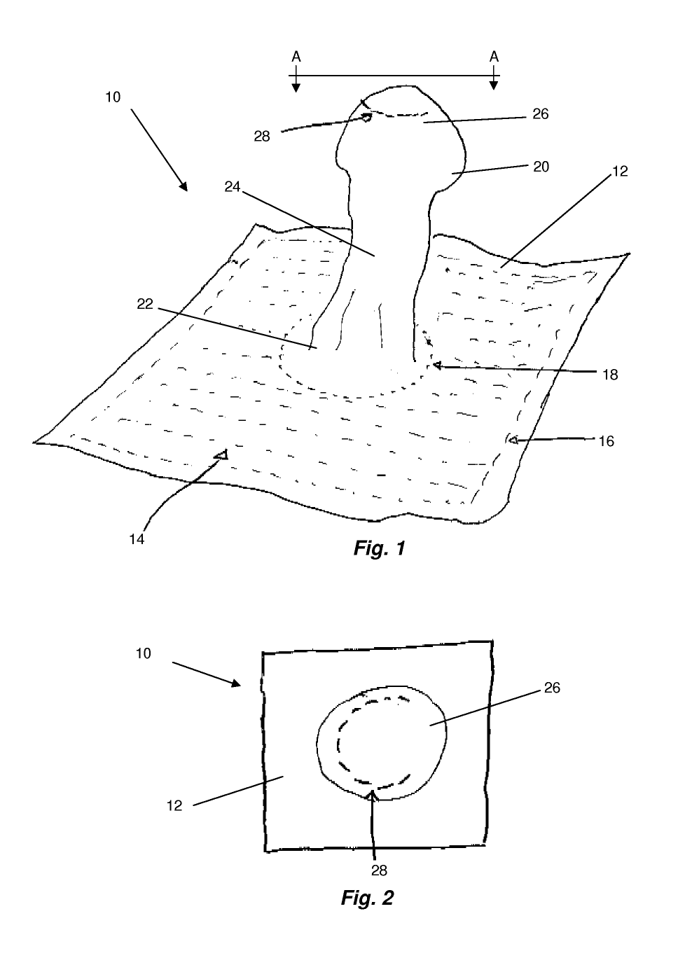

FIG. 1 illustrates a perspective view of a first exemplary embodiment of a surgery cover or drape 10 particularly useful during surgery on the human breast, in particular on the breast of a female human. The cover 10 includes a sheet 12 formed of a flexible material, preferably a flexible polymer material, of a size sufficient to cover portions of the breast of an adult human female, when used in the manner described herein. While FIG. 1 illustrates the sheet 12 being roughly square, the present invention is not so limited, and the sheet can take any shape.

The sheet 12 includes a top surface and a bottom surface, with the bottom surface including an adhesive, mastic, or the like, which permits the sheet to be temporarily adhered to the skin of a patient's breast; those of ordinary skill in the art are well acquainted with adhesives suitable for this purpose, such as those used for bandages and the like. According to an exemplary embodiment of the cover 10, the sheet 12 includes outer 16 and inner 18 regions of the bottom surface of the sheet which are free of the adhesive. The outer region 16 is adhesive-free in order to make it easier for a practitioner to apply and remove the cover 10 to the surgical site, while the inner region 18 is adhesive free so that other portions of the cover, described elsewhere herein, are free to move against the patient's tissues without being inhibited by the adhesive.

The cover 10 further includes a flexible tubular section or tube 20 which extends from the upper surface of the sheet 12 in a first configuration, and which is formed of a material, and is otherwise configured, so that the tube 20 can be evaginated into itself (turned inside out), and thus takes on a second configuration in which the tube extends from the lower surface of the sheet 12. The tube 20 includes a lower, open section 22 at which the tube is attached to the sheet 12, a closed upper section 26 distant from the lower section, and a middle section 24 between the lower and upper sections. While, in the embodiment illustrated in FIG. 1, the lower section 22 is integral with the sheet 12, less preferred (and unillustrated) embodiments include a coupling device between the tube 20 and the sheet 12, so that the tube and the sheet can be attached and detached. Furthermore, the tubular portion of the device can be modified to incorporate “ribs” and/or added stiffness similar to a flexible drinking straw to foster stabilization within the access pathway through the potentially contaminated breast tissue. The length of the tube can be constructed in various sizes in accordance to the surgeon's perceived depth of the breast tissue to be traversed.

The upper section of the tube 20 is capped by a flared dome-like head and is closed. However, it is also constructed so that it can be easily opened by the practitioner. According to an exemplary embodiment, the upper section of the tube includes a portion 28 at which the tube can be ruptured, e.g., by inclusion of a perforation in the material of the tube upper section. Other embodiments include: forming the material of the portion 28 significantly thinner than other, adjacent portions; including a non-perforate tear line in the material; the portion 28 can be readily dissolved by a liquid, e.g., is formed of a water-soluble material; and any other similar mechanism by which the portion 28 can be selectively and controllably, ruptured, broken, or torn. As illustrated in FIG. 2, which is a top plan view of the embodiment of FIG. 1, it is advantageous for the portion 28 to extend over the upper section 26 such that, when the portion 28 is torn or ruptured, the portion 28 forms a flap at the end of the tube 20, rather than completely separating from the tube. As described in detail below, this provision lessens the likelihood that a portion of the cover 10 will be left within the breast tissue of a patient. In the example of FIG. 2, the portion 28 extends roughly 60% along a circular line, although larger and smaller portions are also within the spirit and scope of the present invention. Constructing this device of a colored material, e.g., one that easily contrasts with the tissues in and against which it will be placed, will allow easier identification of any portion of the material inadvertently separated or torn off from the device within the breast cavity.

In addition to the foregoing, other exemplary embodiments can optionally be provided with one or more of the following features. The bottom surface of the sheet 12 can be provided with a release sheet, as is commonplace for adhesively backed items. Antimicrobial, including antibacterial, compounds can be incorporated into the materials of which the cover 10 is formed, or can be coated or otherwise present on some or all of the surfaces of the cover 10. A kit can be provided which includes a sanitary, and preferably sterile, outer enclosure, pouch, tray, or the like, which is constructed to be openable by the practitioner. The enclosure houses at least the cover 10, and optionally includes instructions for the use of the cover, as described herein, which may be either separate from the enclosure (e.g., a piece of paper), printed on the enclosure, or even printed on the cover itself.

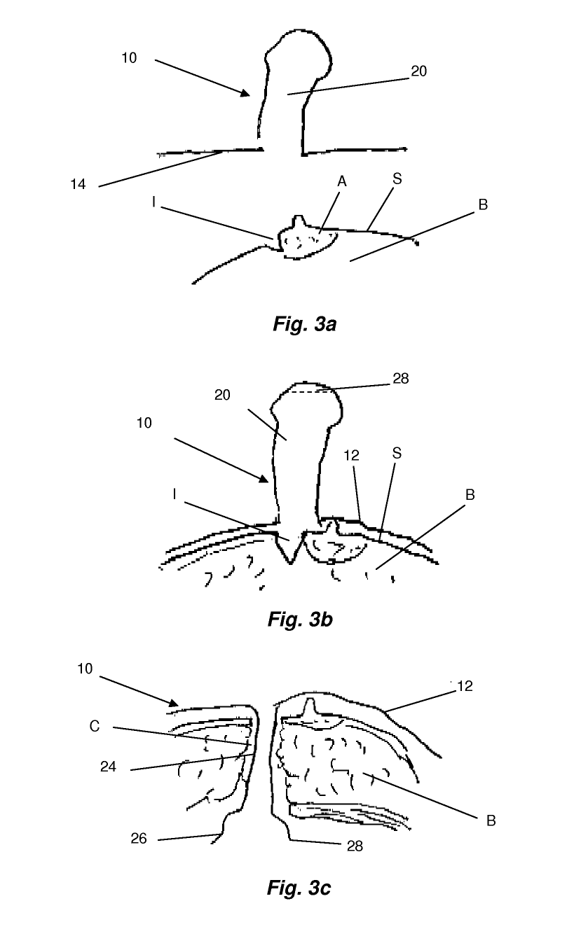

FIGS. 3a-3b illustrate three steps in an exemplary method in accordance with principles of the present invention. At the beginning of a breast implantation surgery, as illustrated in FIG. 3a, an incision I has been formed through the skin S adjacent to the areola A of the breast B of a female human. A cover 10 embodying principles of the present invention is selected for the particular patient, prepared, and is positioned with the tube 20 oriented in the first orientation and away from the incision, and the bottom surface with the adhesive 14 facing the breast tissue.

With reference to FIG. 3b, the cover 10 is then positioned so that the lower, open section 22 is over the incision, and the sheet 12 is pressed down onto the skin S of the breast B. The adhesive 14 thus temporarily attaches the cover 10 to the patient's breast, with the inner region 18, which has no adhesive, overlying and in part surrounding the incision I, and the open end of the tube 20 immediately adjacent and over to the incision. In this manner, the practitioner can effectively seal off the surgical site from the environment, which will inhibit its exposure to bacteria and other pathogens.

With reference to FIG. 3c, the practitioner then evaginates the tube 20, into its second configuration, through the surgical incision and into a cavity C inside the breast tissue in which the implant (not illustrated) will be inserted. The practitioner then manipulates the end 26 of the tube 20, including the rupturable portion 28, to rupture or tear the portion 28, using any mechanism that is expeditious, e.g., by pushing on the portion 28 with the practitioner's finger. Because the cover 10 is held to the patient by the adhesive 14, pushing on the portion 28, when the tube 20 has been put into its second, evaginated configuration, does not risk pushing the cover into the patient, and the portion 28 can be selectively opened. Because, according to certain embodiments, the rupturable portion 28 forms a flap with the tube 20 and does not completely separate from the tube, no portion of the cover can be left inside the patient. Furthermore, the flared domed design of the tube's upper end helps stabilize and secure this end within the cavity, preventing inadvertent extrusion of the tube during implant insertion.

With the end 26 of the tube 20 now adjacent to or in the cavity C, the practitioner can insert an implant (not illustrated) through the open section 22, through the middle section 24, and through the now open section 26 of the tube 20, and into the cavity C. The tube 20 effectively prevents contact of the implant with the patient's skin and with the breast tissue as the implant is pushed into the breast, which can inhibit the introduction of bacteria located on these tissues into the cavity C.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A breast cover useful to inhibit the infection of a female human breast during invasive surgery of the breast, the cover comprising:
a flexible sheet having a first side and a second side, the first side having an adhesive thereon; and
an elongate tube extending from the sheet second side, the tube having a first open end attached to the sheet and a closed second end opposite said first end;
wherein the tube second end is configured and arranged to be controllably ruptured.

2. The cover in accordance with claim 1, wherein the second tube end comprises a perforation.

3. The cover in accordance with claim 1, wherein the tube second end includes a portion which is thinner than adjacent portions of the tube.

4. The cover in accordance with claim 1, wherein the tube second end is water-soluble.

5. The cover in accordance with claim 1, wherein the sheet first side includes a portion adjacent to the elongate tube first end without adhesive.

6. The cover in accordance with claim 1, further comprising:
an antibacterial material positioned on the cover at a location selected from the group consisting of on the sheet first side, on the sheet second side, on the tube inside, on the tube outside, in the material of the sheet, in the material of the tube, and combinations thereof.

7. The cover in accordance with claim 1, wherein the tube is configured and arranged to be evaginated from a first configuration in which said tube extends from the sheet second side, to a second configuration in which the tube extends from said sheet first side.

8. A method of inhibiting a bacterial infection of a female human breast during invasive surgery of said breast, the method comprising:
forming a surgical incision through the skin of the breast;
positioning a cover over the incision, the cover including a tube capable of being evaginated; and
evaginating said tube into said incision.

9. The method according to claim 8, further comprising:
rupturing an end of the tube within the breast tissue.

10. The method according to claim 8, further comprising:
inserting a breast implant through the tube.
